(19) United States
(12) Plant Patent Application Publication
(10) Pub. No.: US 2014/0331373 P1
(43) Pub. Date: Nov. 6, 2014

(54) Osteospermum plant named 'KLEOE12190'

(50) ;

(71) Applicant: Klemm+Sohn GmhH & Co. KG, Stuttgart (DE)

(72) Inventors: Nils Klemm, Stuttgart (DE); Silke Wagener, Leinfelden-Echterdingen (DE)

(73) Assignee: Klemm+Sohn GmbH & Co. KG, Stuttgart (DE)

(21) Appl. No.: 13/986,435

(22) Filed: May 2, 2013

(51) Int. Cl. A01H 5/00

(52) U.S. Cl. PLT/360

(57) ABSTRACT

A new Osteospermum plant particularly distinguished by having violet ray florets and dark green leaves is disclosed.

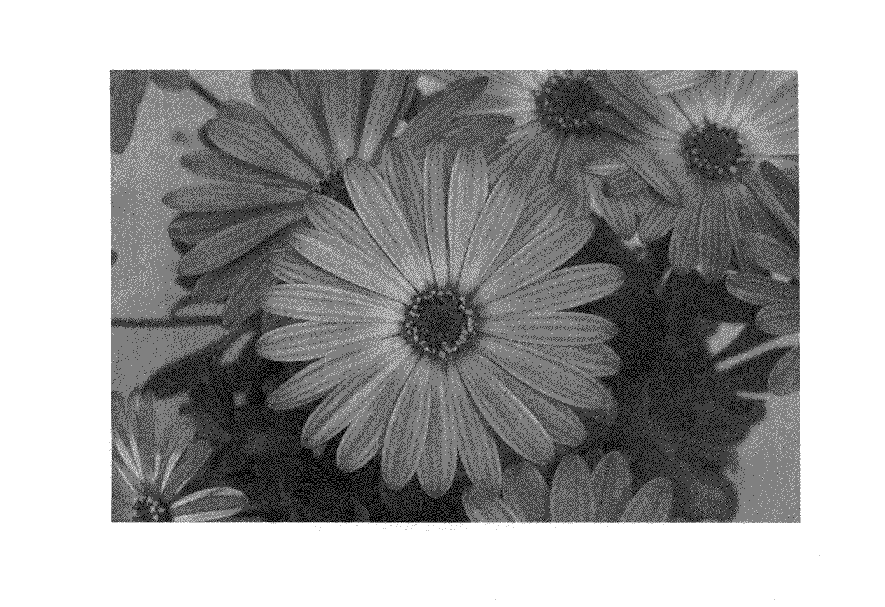

### GENUS AND SPECIES

Osteospermum ecklonis Norl

### VARIETY DENOMINATION

‘KLEOE12190’

### BACKGROUND OF THE NEW PLANT

The present invention comprises a new and distinct variety of Osteospermum, botanically known as Osteospermum ecklonis Norl, and hereinafter referred to by the variety name ‘KLEOE12190’. ‘KLEOE12190’ originated from a cross-pollination between the proprietary female Osteospermum variety ‘SU 10’ (unpatented), having violet ray florets and a light green leaf color, and the proprietary male Osteospermum variety ‘X 95’ (unpatented), having purple to violet ray florets and a dark green flower color.

In the summer of 2008 the two Osteospermum parental lines ‘SU 10’ and ‘X 95’ were crossed in Stuttgart, Germany and seeds were obtained. The seeds were sown and plants were grown for evaluation. A plant line was selected in August 2009 which had violet ray florets and dark green leaves and named ‘KLEOE12190’. In August 2009 ‘KLEOE12190’ was first vegetatively propagated by vegetative cuttings. ‘KLEOE12190’ was found to reproduce true to type in successive generations of asexual propagation via terminal cuttings.

### CROSS REFERENCE TO RELATED APPLICATIONS

Plant Breeder's Rights for ‘KLEOE12190’ were applied for in Canada on February 8, 2013, Application No. 13-7921. ‘KLEOE12190’ has not been made publicly available or sold more than one year prior to the filing date of this application.

### SUMMARY

The following are the most outstanding and distinguishing characteristics of this new variety when grown under normal horticultural practices in Stuttgart, Germany.

1. Violet rays florets; and

2. Dark green leaves.

### DESCRIPTION OF THE PHOTOGRAPH

This new Osteospermum plant is illustrated by the accompanying photograph which shows a close-up of the flowers. The photograph is of a three-month-old plant grown in a controlled greenhouse environment in Stuttgart, Germany under greenhouse conditions in April 2012. The colors shown are as true as can be reasonably obtained by conventional photographic procedures.

### DESCRIPTION OF THE NEW VARIETY

The following detailed descriptions set forth the distinctive characteristics of ‘KLEOE12190’. The data which define these characteristics were collected from asexual reproductions carried out in Stuttgart, Germany. Data was collected on seven-month-old plants grown in a controlled greenhouse environment in Stuttgart, Germany under greenhouse conditions on Jan. 28, 2013. Color references are to The R.H.S. Colour Chart of The Royal Horticultural Society of London (R.H.S.), 5^{th} edition (2007).
Classification:
   Family.—Compositae.
   Botanical.—Osteospermum ecklonis Norl.
   Common.—Osteospermum, African Daisy.
   Designation.—‘KLEOE12190’.
Parentage:
   Female parent.—Osteospermum line ‘SU 10’ (unpatented).
   Male parent.—Osteospermum line ‘X 95’ (unpatented).
Plant:
   Form and habit.—Herbaceous perennial but usually cultivated as an annual.
   Growth habit.—Upright.
   Branching habit.—Freely branching.
   Height (from top of soil, including inflorescence).—15.0 cm.
   Width (including inflorescences).—20.0 cm.
   Propagation.—Terminal cuttings.
   Time to produce a finished flowering plant.—14 weeks.
   Time to initiate and develop roots.—3 weeks.
   Root description.—Freely rooting.
Leaves:
   Arrangement.—Alternate.
   Shape.—Dentiform.
   Apex.—Obtuse.
   Base.—Acute.
   Margin.—Medium.
   Immature color.—Upper surface: RHS 143A Lower surface: RHS 143B.
   Mature color.—Upper surface: RHS 137A Lower surface: RHS 137C.
   Length.—About 7.0 cm.
   Width.—About 4.0 cm.
   Texture.—Upper surface: Leathery, smooth Lower surface: Leathery, shiny.
   Petioles.—Absent.
   Venation pattern.—Pinnate.
   Venation color.—Upper surface: RHS 138B Lower surface: RHS 138B.
Stems:
   Total number of branches per plant (calculated at the base of the plant).—About 5.
   Length.—7.0 cm to 11.0 cm.
   Diameter.—0.6 cm.
   Internode length.—About 1.0 cm.
   Color.—RHS 144B.
   Texture.—Smooth.
   Anthocyanin.—Absent.
Buds:
   Shape.—Obovate.
   Length.—About 1.0 cm.
   Diameter.—About 1.0 cm.
   Color (at tight bud just before the ray florets unfold): RHS 143A.
Inflorescence:
   Type.—Single.
   Blooming habit.—Spring and autumn.
   Quantity of inflorescences per plant.—20 to 30.
   Lastingness of the inflorescences on the plant.—About 2 weeks.
   Fragrance.—Absent to faint.
   Inflorescence diameter.—About 5.5 cm.
   Inflorescence depth.—About 1.0 cm.
   Disc diameter.—1.5 cm.
Disc floret:
   Quantity per inflorescence.—About 120.
   Shape.—Lanceolate.
   Tube color.—Closed: RHS 138B Mature: RHS 83B.
   Length.—0.6 cm.
   Diameter (at apex).—0.1 cm.
   Apex.—Obtuse.
   Apex color.—RHS N88A.
   Base.—Acute.
   Base color.—RHS 145C.
   Margin.—Entire.
Ray floret:
   Quantity per inflorescence.—About 26.
   Shape.—Oblanceolate.
   Color.—Upper surface: RHS N87C Lower surface: RHS N87D.
   Length.—2.2 cm.
   Width.—0.6 cm.
   Apex.—Obtuse.
   Base.—Acute.
   Margin.—Entire.
   Texture.—Upper surface: Smooth Lower surface: Smooth, shiny.
   Peduncle.—Length: 2.0 cm to 2.5 cm Diameter: 0.2 cm Texture: Rough Color: RHS 143B.
Phyllaries:
   Texture.—Smooth.
   Color.—RHS 143A.
   Arrangement.—Single.
   Observed quantity per plant.—1 to 20.
   Shape.—Lanceolate.
   Color.—Upper surface: RHS 143A Lower surface: RHS 143A.
   Length.—1.2 cm.
   Width.—0.2 cm.
   Apex.—Acute.
   Base.—Acute.
   Margin.—Entire.
   Texture.—Upper surface: Smooth Lower surface: Smooth, rough.
Reproductive organs:
   Androecium.—Location: On the disc floret Stamens: Quantity: Numerous Shape: Filamentous Filament length: 0.5 cm Filament diameter: Less than 0.05 cm Anther: Shape: Lanceolate Color: RHS N92A Length: 0.2 cm Diameter: Less than 0.05 cm Pollen: Color: RHS 12A Amount: Moderate.
   Gynoecium.—Location: On the base of the petals (ray floret) Pistils: Number: About 26 Length: 0.4 cm Diameter: Less than 0.05 cm.
   Stigma.—Color: RHS 83A Shape: Tapering, conical Length: 0.2 cm Diameter: Less than 0.05 cm.
   Style.—Color: RHS 75B Length: 0.2 cm Diameter: Less than 0.05 cm.
Fruit and seed set: None observed
Disease and pest/insect resistance: No particular disease or insect resistance observed.

### COMPARISON WITH PARENTAL LINES AND KNOWN VARIETY

‘KLEOE12190’ is a distinct variety of Osteospermum having violet ray florets and dark green leaves; ‘KLEOE12190’ is distinguished from its parents as described in Table 1:

TABLE 1
Comparison with Parental Lines

| | | Female parent | Male Parent |
|---|---|---|---|
| | | ‘SU 10’ | ‘X 95’ |
| Trait | ‘KLEOE12190’ | (unpatented) | (unpatented) |
| Ray floret color | Violet | Violet | Purple to violet |
| Color of leaves | Dark green | Lighter green | Dark green |

‘KLEOE12190’ is a distinct variety of Osteospermum having violet ray florets and dark green leaves. ‘KLEOE12190’ is most similar to the Osteospermum plant named ‘FlowerPower Purple Blue’ (unpatented). Differences between the two varieties are described in Table 2:

TABLE 2
Comparison with Similar Variety

| | | Commercial line |
|---|---|---|
| | | ‘FlowerPower Purple Blue’ |
| Trait | ‘KLEOE12190’ | (unpatented) |
| Color of ray florets | Violet | Purple-violet |

We claim:

1. A new and distinct variety of Osteospermum plant named ‘KLEOE12190’ as shown and described herein.
